(19) United States
(12) Patent Application Publication
(10) Pub. No.: US 2024/0398874 A1
(43) Pub. Date: Dec. 5, 2024

(54) COMPOSITION FOR TREATMENT OF WOUNDS CONTAINING STEM CELL-DERIVED EXOSOMES AND METHOD FOR PREPARING SAME

(71) Applicant: BREXOGEN INC., Seoul (KR)

(72) Inventors: Soo KIM, Seoul (KR); Minyoung Jung, Seoul (KR)

(73) Assignee: BREXOGEN INC., Seoul (KR)

(21) Appl. No.: 18/677,400

(22) Filed: May 29, 2024

(30) Foreign Application Priority Data
Jun. 1, 2023 (KR) 10-2023-0070622

(51) Int. Cl. A61K 35/545; A61P 17/02; C12N 5/0775

(52) U.S. Cl. CPC A61K 35/545; A61P 17/02; C12N 5/0662; C12N 2501/905; C12N 2506/45

(57) ABSTRACT

Disclosed are a composition containing stem cell-derived exosomes for treatment of wounds and a method for preparing the same, wherein the exosomes according to the present disclosure can be used as an active ingredient of a pharmaceutical composition for treatment of wounds due to excellent wound healing promoting effect.

### CROSS-REFERENCE TO RELATED APPLICATIONS

This application claims priority to Korean Patent Application No. 10-2023-0070622 filed on Jun. 1, 2023, the entire disclosures of which are incorporated herein by reference.

### BACKGROUND OF THE INVENTION

### 1. Field of the Invention

The present disclosure relates to a composition for treatment of wounds containing stem cell-derived exosomes and a method for preparing the same and, more specifically, to a composition containing exosomes, which are isolated from mesenchymal stem cells or a culture thereof and are confirmed to have excellent wound healing promoting effects through experiments using human dermal fibroblasts and animal models, thereby treating or preventing wounds.

### 2. Description of the Prior Art

Extracellular vesicles are vesicles composed of spherical lipid-bilayers with a size of 30-1000 nm, including microvesicles and exosomes.

Exosomes have lipid bilayers that have the same phospholipid bilayer structure as in the origin cells (donor cells) thereof, and are compositions of substances extracellularly released by the cells. Exosomes are known to perform functional roles, such as cell-cell communication and cellular immune intervention.

Exosomes carry cell-specific constituents accounting for biological functions, which are characteristic of origin cells and contain various water-soluble proteins, peripheral proteins, and transmembrane proteins in addition to phospholipids, mRNA, and miRNA.

These exosomes are excreted from all animal cells, such as mast cells, lymphocytes, astrocytes, platelets, nerve cells, endothelial cells, and epithelial cells, and are found in various body fluids including blood, urine, mucus, saliva, bile juice, ascitic fluid, cerebrospinal fluid, and so on. Exosomes exhibit highly selective penetration sufficient to cross even the blood-brain barrier (BBB) as well as cell membranes of epidermal and endothelial cells, so that exosomes can find applications in the development of drug delivery systems (DDS) utilizing nano-carriers for specific drugs.

Exosomes and microvesicles released from mesenchymal stem cells are involved in cell-to-cell communication and show medicinally regenerative therapeutic efficacy that stem cells possess.

Exosomes are known to bring about a trophic effect into paracrine factors secreted from stem cells that have been transplanted in vivo without survival for a long period of time. Such factors as small molecules like growth factors, chemokines, and cytokines are released by extracellular vesicles such as exosomes, which are derived from stem cells. Therefore, exosomes are utilized for characterizing stem cells and evaluating therapeutic efficacies thereof.

In recent years, active research into therapeutic effects of exosomes secreted by mesenchymal stem cells, but not mesenchymal stem cells themselves, on various diseases have been ongoing. In academia and industrial fields, it is expected that this strategy might be a new promising alternative that can surmount the limitation of conventional stem cell therapies.

### SUMMARY OF THE INVENTION

The present inventors developed a composition containing exosomes isolated from stem cells or a culture thereof, and identified that the composition according to the present disclosure exhibits an excellent wound healing promoting effect as a result of experiments using human dermal fibroblasts and burn-induced animal models, and thus found that the treatment of wounds can be attained through the composition of the present disclosure.

Accordingly, an aspect of the present disclosure is to provide a composition containing stem cell-derived exosomes.

Another aspect of the present disclosure is to provide a pharmaceutical composition for treatment of wounds containing stem cell-derived exosomes.

Still another aspect of the present disclosure is to provide a method for treating wounds using stem cell-derived exosomes.

Still another aspect of the present disclosure is to provide use of a composition containing stem cell-derived exosomes for the treatment of wounds.

Still another aspect of the present disclosure is to provide a wound dressing material containing stem cell-derived exosomes.

Still another aspect of the present disclosure is to provide a method for preparing a pharmaceutical composition for treatment of wounds, the pharmaceutical composition containing stem cell-derived exosomes.

The present disclosure is directed to a composition for treatment of wounds containing stem cell-derived exosomes and a method for preparing the same, and wounds can be treated through the composition of the present disclosure.

Hereinafter, the present disclosure will be described in more detail.

In accordance with an aspect of the present disclosure, there is provided a composition containing stem cell-derived exosomes.

As used herein, the term “exosomes” refers to cell-derived vesicles, which are found in body fluids of almost all eukaryotic organisms and have a diameter of about 30-100 nm, larger than LDL proteins but far smaller than erythrocytes. When multivesicular bodies (MVBs) are fused to cell membranes, exosomes are secreted from cells or immediately from the cell membranes. Such exosomes are well known to perform important and specialized functions, such as coagulation and cell-to-cell signaling.

As used herein, the term “stem cells” refers to undifferentiated cells that can self-renew and differentiate into two or more different types of cells.

In an embodiment of the present disclosure, the stem cells may be autogenous or homogenous stem cells, may be originated from any type of animals including humans and non-human mammals, may be derived from adults, or may be derived from embryos. For example, the stem cells may be selected from the group consisting of adult stem cells, embryonic stem cells, induced pluripotent stem cells (iPSCs), iPSC-derived mesenchymal stem cells, BxC stem cells, and hyaluronic acid (HA)-pretreated iPSC-derived mesenchymal stem cells, and BxC-HA stem cells, but are not limited thereto.

As used herein, the term “adult stem cells” refers to undifferentiated cells just before differentiation to cells of specific organs, found in cord blood, adult bone marrow, blood, etc., which has the ability to develop to tissues of the body as needed.

In an embodiment of the present disclosure, the adult stem cells may be selected from the group consisting of adult stem cells originated from humans, animals, or animal tissues, mesenchymal stromal cells originated from humans, animals, or animal tissues, and mesenchymal stromal cells and multipotent stem cells derived from induced pluripotent stem cells originated from human, animal, or animal tissues, but are not limited thereto.

In the present disclosure, the human, animal, or animal tissue may be selected from the group consisting of umbilical cord, cord blood, bone marrow, fat, muscle, nerve, skin, amniotic membrane, and placenta, but is not limited thereto.

The stem cells originated from various tissues of humans or animals may be selected from the group consisting of hematopoietic stem cells, mammary stem cells, intestinal stem cells, vascular endothelial progenitor cells, neural stem cells, olfactory neural stem cell, and testicular stem cells, but are not limited thereto.

As used herein, the term “embryonic stem cells” refers to cells isolated during the embryogenesis, which is derived from the inner cell mass of a blastocyst formed prior to implantation of the fertilized egg in the uterus, and cultured in vitro.

Embryonic stem cells are cells that are pluripotent or totipotent and give rise to differentiation to cells of all tissues and exhibit self-renewal, and encompass embryoid bodies (EBs) derived therefrom in a wide sense.

In the present disclosure, the embryonic stem cells may include embryonic stem cells derived from all origins, such as humans, monkeys, pigs, horses, cattle, sheep, dogs, cats, mice, and rabbits, but with no limitations thereto.

As used herein, the term “induced pluripotent stem cell” or “iPSCs” refers to pluripotent cells derived from differentiated cells through artificial dedifferentiation and may be interchangeably used with “dedifferentiated stem cell”.

The artificial dedifferentiation may be conducted by introducing dedifferentiation factors through viral mediation using retroviruses, lentiviruses, and Sendai viruses, through non-viral mediation with the aid of non-viral vectors, proteins, and cell extracts, or through stem cell extracts, compounds, or the like.

The induced pluripotent stem cells exhibit almost the same characteristics as embryonic stem cells. Specifically, the induced pluripotent stem cells are common in the aspects including cellular morphological similarity, similar gene and protein expression pattern, pluripotency in vitro and in vivo, teratoma formation, creation of chimeric mice upon insertion into murine blastocysts, and germline transmission of genes.

In an embodiment of the present disclosure, the stem cells may be induced pluripotent stem cell-derived mesenchymal stem cells.

As used herein, the term “mesenchymal stem cells” or “MSCs” refers to stem cells derived from the mesenchyma. The mesenchymal stem cells may differentiate into at least one selected from the group consisting of osteoblasts, chondrocytes, adipocytes, and myocytes. The mesenchymal stem cells may be isolated from any type of adult tissues, for examples, from bone marrow, adipose tissue, umbilical cord, or peripheral blood. Mesenchymal stem cell (MSC) populations are defined by their characteristic phenotypes. Mesenchymal stem cell populations differentiated from induced pluripotent stem cells may exhibit the same phenotype as in typical mesenchymal stem cell populations, which may be understood to express CD105, CD73, and CD90 markers at a level of 95% or higher and CD45, CD34, and SSEA-4 at a level of 5% or less, 3% or lower, 2% or lower, or 1% or lower. Particularly, cells expressing at most 5%, 3%, 2%, or 1% of CD45, CD34, or SSEA-4 may, for convenience, be expressed as “not expressing” CD45, CD34, or SSEA-4.

In an embodiment of the present disclosure, the composition may contain induced pluripotent stem cell-derived mesenchymal stem cell-derived exosomes.

In an embodiment of the present disclosure, the stem cells may be BxC stem cells.

As used herein, the term “BxC stem cells” refers to stem cell prepared by culturing induced pluripotent stem cells, isolating a population of induced pluripotent stem cells (iPSCs) not expressing stage-specific embryonic antigen 4 (SSEA-4) protein, and then further culturing the population of stem cells. The BxC stem cells are cells in the stage immediately before complete differentiation from induced pluripotent stem cells into mesenchymal stem cells, and can have the properties of complete mesenchymal stem cells through additional culturing. Therefore, the BxC stem cell population does not exhibit completely identical phenotype to that of the mesenchymal stem cell population, and may be similar in phenotype to the mesenchymal stem cell population in the range of 96% to 99.9%. For instance, CD90 protein may be expressed at a level of 0.3% in the induced pluripotent stem cell population, at a level of 99.7% in the mesenchymal stem cell population, and at a level of 96.9% in the BxC stem cell population, which amounts to about 98% of the expression level in the mesenchymal stem cells. Consequently, BxC stem cells may be defined as stem cells into which the induced pluripotent stem cells, obtained as stem cells not expressing SSEA-4 proteins upon culturing induced pluripotent stem cells, are differentiated at 96% to 99.9% but are not completely differentiated into mesenchymal stem cells.

The BxC stem cells are superior in terms of stemness to mesenchymal stem cells differentiated from the same induced pluripotent stem cells, and can secrete a large amount of functionality-related proteins. Specifically, the BxC stem cells of the present disclosure are 10 or more times greater in proliferative capacity than mesenchymal stem cells derived from the same tissue after undergoing 9 passages and are not observed to decrease in proliferative capacity even after 12 or more passages. In addition, the expression level of Ki67, a marker related to cell proliferative capacity, is more than twice higher in BxC stem cells than in general mesenchymal stem cells. Compared with mesenchymal stem cells differentiated from the same induced pluripotent stem cells, the BxC stem cells can express a higher level of at least one gene selected from the group consisting of ANKRD1, CPE, NKAIN4, LCP1, CCDC3, MAMDC2, CLSTN2, SFTA1P, EPB41L3, PDE1C, EMILIN2, SULT1C4, TRIM58, DENND2A, CADM4, AIF1L, NTM, SHISA2, RASSF4, and ACKR3 and a lower level of at least one gene selected from the group consisting of DHRS3, BMPER, IFI6, PRSS12, RDH10, and KCNE4.

As used herein, the term “stemness” refers to the capability for pluripotency of differentiating into all types of cells and for self-renewal of dividing indefinitely to produce more of the same stem cell. For example, stemness indicates the capabilities for increasing proliferation of stem cells while maintaining stem cells in an undifferentiated state, for increasing telomerase activity, for increasing expression of stemness acting signals, and for increasing cell mobility, and may encompass exhibiting at least one of these capabilities.

In an embodiment of the present disclosure, the composition may contain BxC stem cell-derived exosomes.

In an embodiment of the present disclosure, the stem cells may be hyaluronic acid-pretreated induced pluripotent stem cell-derived mesenchymal stem cells.

As used herein, the term “pretreatment” refers to a process of culturing mesenchymal stem cells in a medium containing a specific material. For example, the pretreatment means a process in which mesenchymal stem cells completely differentiated from iPSCs are further cultured in a medium containing hyaluronic acid. In the present disclosure, hyaluronic acid can enhance the stemness and proliferation ability of stem cells and increase the number of stem cell-derived exosomes and the content of proteins and RNA in exosomes.

In an embodiment of the present disclosure, the composition may contain exosomes isolated from hyaluronic acid-pretreated-induced pluripotent stem cell-derived mesenchymal stem cells.

In an embodiment of the present disclosure, the stem cells may be hyaluronic acid-pretreated BxC stem cells.

As used herein, the term “hyaluronic acid-pretreated BxC stem cells” refers to mesenchymal stem cells obtained by culturing BxC stem cells according to the present disclosure to completely differentiate into mesenchymal stem cells and then culturing (pretreating) the mesenchymal stem cells in a medium containing hyaluronic acid. For instance, the hyaluronic acid-pretreated BxC stem cells may be prepared by further culturing BxC stem cells to completely differentiate into mesenchymal stem cells and then culturing the mesenchymal stem cells in a medium containing hyaluronic acid at 0.1 to 1000 ug/mL, for example, 40 ug/mL for 12 to 48 hours. The hyaluronic acid-pretreated BxC stem cells increase the proliferative capacity by about 360%, exosome productivity by about 5-fold, and exosome-derived protein levels by at least 5-fold, compared with the mesenchymal stem cells treated with no substances.

In an embodiment of the present disclosure, the treatment may be carried out with hyaluronic acid at a concentration of 0.1 to 1000 ug/mL, 0.5 to 1000 ug/mL, 1 to 500 ug/mL, 1 to 200 ug/mL, 1 to 100 ug/mL, 1 to 80 ug/mL, 1 to 60 ug/mL, or 10 to 60 ug/mL, for example, 40 ug/mL, but is not limited thereto.

In accordance with another aspect of the present disclosure, there is provided a pharmaceutical composition for treatment of wounds, the pharmaceutical composition containing, as an active ingredient, exosomes isolated from induced pluripotent stem cell-derived mesenchymal stem cells.

As used herein, the term “wound” refers to a state in which the body tissue is injured, and may refer to the injury to the skin, subcutaneous tissue, muscles, nerves, vessels, organ tissues, or the like. Examples of the wound may include contusions, abrasions, lacerations, incisions, punctures, burns, frostbites, or abnormal protrusions, invaginations, or scars formed in tissues due to such injuries, but are not limited thereto.

As used herein, the term “treatment” refers to inhibiting the development, alleviating, or eliminating disorders, diseases, or symptoms. The composition according to an embodiment of the present disclosure significantly increases wound healing rates (FIGS. 1 to 3), promotes elasticity or collagen fiber reproduction in wound areas (FIGS. 4 and 5), and promotes the division of human dermal fibroblasts and the expression of genes related to wound healing (FIGS. 6 to 12), in wound-induced animal models. Therefore, the composition according to an embodiment of the present disclosure can exhibit both a wound treatment effect and a wound treatment promoting effect.

As used herein, the term “containing as an active ingredient” means containing a sufficient amount to achieve a specific effect, for example, activity in the treatment or prevention of wound, rapid promotion of wound healing, restoration of extracellular environments, of exosomes isolated from stem cells or a culture thereof.

The pharmaceutical composition of the present disclosure may further contain a pharmaceutically acceptable carrier.

As used herein, the term “pharmaceutically acceptable” refers to being commonly usable in the field of pharmacy, without inhibiting biological activity and characteristics of a compound to be administered, while causing no stimulation to an organism.

In the present disclosure, any carrier that is commonly used in the art may be used. Non-limiting examples of the carrier may be a saline solution, sterile water, Ringer's solution, a buffered saline solution, an albumin injection solution, lactose, dextrose, sucrose, sorbitol, mannitol, xylitol, erythritol, maltitol, maltodextrin, glycerol, ethanol, or a combination thereof.

In the present disclosure, the pharmaceutical composition may be used by addition of other pharmaceutically acceptable additives, such as an excipient, a diluent, an antioxidant, a buffer, or a bacteriostat, if necessary, and furthermore, a filler, an extender, a humectant, a disintegrant, a dispersant, a surfactant, a binder, a lubricant, and the like may be additionally used.

In an embodiment of the present disclosure, the induced pluripotent stem cell-derived mesenchymal stem cells may be differentiated from progenitor cells of induced pluripotent stem cell-derived mesenchymal stem cells not expressing stage-specific embryonic antigen 4 (SSEA-4).

In an embodiment of the present disclosure, the induced pluripotent stem cells may be human-derived induced pluripotent stem cells.

In an embodiment of the present disclosure, the induced pluripotent stem cell-derived mesenchymal stem cells may be pretreated with a pretreatment substance.

In an embodiment of the present disclosure, the pretreatment substance may be hyaluronic acid.

In accordance with still another aspect of the present disclosure, there is provided a wound treatment method including:

providing exosomes, isolated from induced pluripotent stem cell-derived mesenchymal stem cells, to a subject through administration or contact.

As used herein, the term “subject” may be a mammal including a human, and examples thereof may include humans, monkeys, cows, horses, sheep, pigs, chickens, turkeys, quails, cats, dogs, mice, rats, rabbits, or guinea pigs, but are not limited thereto. As used herein, the term “administration” refers to provision of a predetermined substance to a patient in an appropriate manner, and the composition containing exosomes of the present disclosure may be administered orally or parenterally via all general routes so long as the composition can reach a target tissue. The composition of the present disclosure may be administered using any apparatus that can deliver the active ingredient to a target cell.

As used herein, the term “contact” refers to provision of a predetermined substance to a subject by any appropriate method that is non-invasive to the body. For example, the composition containing exosomes of the present disclosure may be brought into contact with the subject so that the composition can be directly contacted or indirectly delivered to the epidermis or outer skin of the subject.

In an embodiment of the present disclosure, the exosomes may include hyaluronic acid-pretreated BxC stem cell-derived exosomes (BxC-Exo).

In accordance with still another aspect of the present disclosure, there is provided use of a pharmaceutical composition for treatment of wounds, the pharmaceutical composition containing, as an active ingredient, exosomes isolated from induced pluripotent stem cell-derived mesenchymal stem cells.

In accordance with still another aspect of the present disclosure, there is provided a wound dressing material containing exosomes isolated from induced pluripotent stem cell-derived mesenchymal stem cells.

As used herein, the term “wound dressing material” refers to a medical device used to cover a wound lesion to prevent the contamination of the wound lesion or protect the wound lesion from an additional injury and to absorb blood or body fluids flowing out from the wound area. The wound dressing material have any form that can cover a wound area, for example, a gel type, a film type, a foam type, or a hydrocolloid type, but is not limited thereto.

The wound dressing material according to an embodiment of the present disclosure can prevent the contamination or injury to the wound lesion and promote the healing of the wound lesion by containing the stem cell-derived exosomes according to an embodiment of the present disclosure or a composition containing the same.

In an embodiment of the present disclosure, the induced pluripotent stem cell-derived mesenchymal stem cells may be differentiated from progenitor cells of induced pluripotent stem cell-derived mesenchymal stem cells not expressing SSEA-4 protein.

In an embodiment of the present disclosure, the induced pluripotent stem cells may be human-derived induced pluripotent stem cells.

In an embodiment of the present disclosure, the induced pluripotent stem cell-derived mesenchymal stem cells may be pretreated with a pretreatment substance.

In an embodiment of the present disclosure, the pretreatment substance may be hyaluronic acid.

In accordance with still another aspect of the present disclosure, there is provided a method for preparing a pharmaceutical composition for treatment of wounds, the method including:
a first culturing step of culturing induced pluripotent stem cells in a medium;
a selective culturing step of separating SSEA-4(−) cells not expressing SSEA-4 protein from the cultured induced pluripotent stem cells and culturing the SSEA-4(−) cells to allow the SSEA-4(−) cells to differentiate into BxC stem cells;
a second culturing step of culturing BxC stem cells to allow the BxC stem cells to differentiate into mesenchymal stem cells;
a pretreatment step of pretreating the mesenchymal stem cells with hyaluronic acid;
a producing step of culturing the pre-treated mesenchymal stem cells to produce exosomes; and
an isolating step of isolating exosomes from the mesenchymal stem cells or a culture thereof.

In an embodiment of the present disclosure, in the first culturing step, the induced pluripotent stem cells may be cultured in a medium containing fetal bovine serum (FBS) and basic fibroblast growth factor (bFGF) for 1 to 10 days.

In an embodiment of the present disclosure, the induced pluripotent stem cells may be prepared by culturing somatic cells, derived from umbilical cord, cord blood, bone marrow, fat, muscle, nerve, skin, amniotic membrane, amniotic fluid, or placenta of humans, in a dedifferentiation medium. Specifically, the somatic cells from which induced pluripotent stem cells are differentiated may include fibroblasts, hepatocytes, adipose cells, epithelial cells, epidermal cells, chondrocytes, muscle cells, cardiac muscle cells, melanocytes, neural cells, glial cells, astroglial cells, monocytes, macrophages, or mesenchymal stem cells derived from bone marrow, umbilical cord, cord blood, adipose tissue, amniotic fluid, or tooth buds of molar teeth, but are not limited thereto.

In an embodiment of the present disclosure, in the selective culturing step, SSEA-4(−) cells not expressing SSEA-4 protein, separated from the induced pluripotent stem cells, may be differentiated into BxC stem cells by culturing in a medium containing FBS and bFGF for 1-10 days.

In an embodiment of the present disclosure, the pretreatment step may include a step of culturing the mesenchymal stem cells in a medium containing hyaluronic acid at a concentration of 0.1 to 1000 μg/ml, 0.5 to 1000 μg/ml, 1 to 500 μg/ml, 1 to 200 μg/ml, 1 to 100 μg/ml, 1 to 80 μg/ml, 1 to 60 μg/ml, or 10 to 60 μg/ml, for example, at a concentration of 40 μg/ml, but is not limited thereto.

In an embodiment of the present disclosure, the producing step may further include an additional culturing step of culturing the mesenchymal stem cells in exosome-free FBS. Unlike general FBS containing a large amount of bovine serum-derived exosomes, exosome-free FBS is FBS with exosomes removed therefrom and can prevent the incorporation of FBS-derived exosomes into the medium instead of the exosomes secreted from stem cells.

In the present disclosure, the cell culture medium may be any medium for culturing stem cells that is commonly used in the art, and for example, DMEM (Dulbecco's Modified Eagle's Medium), MEM (Minimal Essential Medium), BME (Basal Medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-MEM (α-Minimal essential Medium), G-MEM (Glasgow's Minimal Essential Medium), IMDM (Isocove's Modified Dulbecco's Medium), KnockOut DMEM, and E8 (Essential 8 Medium), which are commercially available or artificially synthetic media, may be used, but the cell culture medium is not limited thereto. The cell culture medium may further contain an ingredient, such as a carbon source, a nitrogen source, a trace element, an amino acid, an antibiotic, and the like.

In the present disclosure, in the isolation step, the culture medium of the stem cells is centrifuged at 200-400×g for 5 to 20 minutes to remove remaining cells and cell debris, and thereafter, the supernatant is taken and subjected to high-speed centrifugation at 9,000-12,000×g for 60-80 minutes, and then the supernatant is again taken and subjected to centrifugation at 90,000-120,000×g for 80-100 minutes to remove the resultant supernatant, thereby obtaining exosomes remaining in the bottom layer.

The present disclosure is directed to a composition for treatment of wounds, the composition containing stem cell-derived exosomes, and a method for preparing the same, and the exosomes according to the present disclosure can be used as an active ingredient of a pharmaceutical composition for treatment of wounds due to excellent wound healing promoting effect.

### BRIEF DESCRIPTION OF THE DRAWINGS

The above and other aspects, features and advantages of the present disclosure will be more apparent from the following detailed description taken in conjunction with the accompanying drawings.

FIG. 1 shows images obtained by observing the degree of wound healing over time after the treatment with exosomes (BxC-Exo) isolated from hyaluronic acid-pretreated induced pluripotent stem cell-derived mesenchymal stem cells in burn injury-induced animal models according to an example of the present disclosure.

FIG. 2 shows a graph comparing the degree of wound healing over time after the treatment with exosomes (BxC-Exo) isolated from hyaluronic acid-pretreated induced pluripotent stem cell-derived mesenchymal stem cells in burn injury-induced animal models according to an example of the present disclosure.

FIG. 3 shows images obtained by microscopic observation of skin tissues from the wound lesions that underwent special staining 15 days after the treatment with exosomes (BxC-Exo) isolated from hyaluronic acid-pretreated induced pluripotent stem cell-derived mesenchymal stem cells in burn injury-induced animal models according to an example of the present disclosure.

FIG. 4 shows a graph depicting the relative density, compared with the control group, of elastin fibers in the wound lesion 15 days after the treatment with exosomes (BxC-Exo) isolated from hyaluronic acid-pretreated induced pluripotent stem cell-derived mesenchymal stem cells in burn injury-induced animal models according to an example of the present disclosure.

FIG. 5 shows a graph depicting the relative density, compared with the control group, of collagen fibers in the wound lesion 15 days after the treatment with exosomes (BxC-Exo) isolated from hyaluronic acid-pretreated induced pluripotent stem cell-derived mesenchymal stem cells in-induced animal models according to an example of the present disclosure

FIG. 6 shows images comparing, compared with the control group, wound healing 24 hours after the injured human dermal fibroblasts were treated with exosomes (BxC-Exo), isolated from hyaluronic acid-pretreated induced pluripotent stem cell-derived mesenchymal stem cells, at different concentrations (1, 5, and 10% v/v) according to an example of the present disclosure.

FIG. 7 shows a graph comparing, compared with the control group, wound healing rates 24 hours after the injured human dermal fibroblasts were treated with exosomes (BxC-Exo), isolated from hyaluronic acid-pretreated induced pluripotent stem cell-derived mesenchymal stem cells, at different concentrations (1, 5, and 10% v/v) according to an example of the present disclosure.

FIG. 8 shows a graph comparing, compared with the control group, the expression level of the epidermal growth factor (EGF) gene in human dermal fibroblasts treated with exosomes (BxC-Exo), isolated from hyaluronic acid-pretreated induced pluripotent stem cell-derived mesenchymal stem cells according to an example of the present disclosure.

FIG. 9 shows a graph comparing, compared with the control group, the expression level of the insulin-like growth factor 1 (IGF1) gene in human dermal fibroblasts treated with exosomes (BxC-Exo), isolated from hyaluronic acid-pretreated induced pluripotent stem cell-derived mesenchymal stem cells according to an example of the present disclosure.

FIG. 10 shows a graph comparing, compared with the control group, the expression level of the elastin (ELN) gene in human dermal fibroblasts treated with exosomes (BxC-Exo), isolated from hyaluronic acid-pretreated induced pluripotent stem cell-derived mesenchymal stem cells according to an example of the present disclosure.

FIG. 11 shows a graph comparing, compared with the control group, the expression level of the elastin (FN1) gene in human dermal fibroblasts treated with exosomes (BxC-Exo), isolated from hyaluronic acid-pretreated induced pluripotent stem cell-derived mesenchymal stem cells according to an example of the present disclosure.

FIG. 12 shows a graph comparing, compared with the control group, the expression level of the collagen type 1-A1 (COL1A1) gene in human dermal fibroblasts treated with exosomes (BxC-Exo), isolated from hyaluronic acid-pretreated induced pluripotent stem cell-derived mesenchymal stem cells according to an example of the present disclosure.

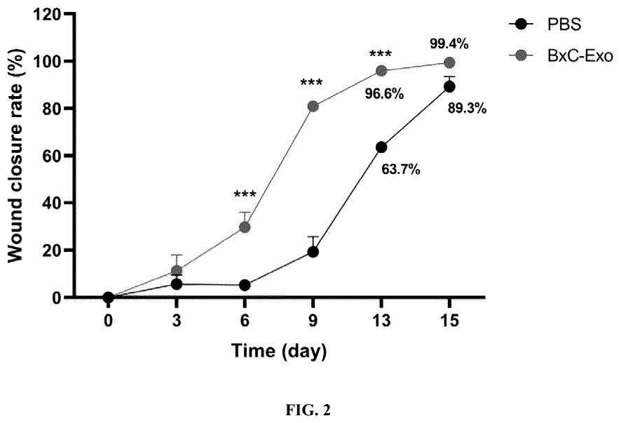

In FIGS. 2, 4, 5, and 7 to 12, * (p<0.05), ** (p<0.01), ***(p<0.001) in each graph.

### DETAILED DESCRIPTION OF THE EXEMPLARY EMBODIMENTS

Hereinafter, the present disclosure will be described in more detail by the following exemplary embodiments. However, these exemplary embodiments are used only for illustration, and the scope of the present disclosure is not limited by these exemplary embodiments.

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

Unless otherwise specified, all numbers, values and/or expressions expressing components, reaction conditions and the content of an ingredient, which are used herein, are approximate values that reflect various uncertainties of measurement occurring to obtain these values among essentially different things, and thus in all cases, it should be understood that the values are modified by the term “about”.

In addition, when a numerical range is specified in the specification, such a range is continuous, and unless indicated otherwise, includes all values from the minimum value to the maximum value of the range.

Additionally, the term “or” as used herein is intended to mean inclusive “or” but not exclusive “or”. In other words, when a combination or utilization between components is not otherwise specified or not contextually clear, that is, when X includes A; X includes B; or X includes both A and B, “X includes A or B” can be applied to any of these.

### Example 1: Culturing of Induced Pluripotent Stem Cell-Derived Mesenchymal Stem Cells

Induced pluripotent stem cells (iPSCs) were cultured for 7 days in DMEM supplemented with 10% fetal bovine serum (FBS) and 10 ng/ml bFGF. Thereafter, SSEA-4(−) and CD34(−) cells not expressing stage-specific embryonic antigen 4 (SSEA-4) and cluster of differentiation 34 (CD34) proteins on the surfaces thereof were separated by FACS analysis from the cultured induced pluripotent stem cells to obtain progenitor cells of induced pluripotent stem cell-derived mesenchymal stem cells. Subsequently, the separated SSEA-4(−) cells were passaged and cultured for additional seven days in DMEM supplemented with 10% FBS and 10 ng/ml bFGF to produce BxC stem cells.

Thereafter, a comparison was made of the expression levels of functional proteins between the produced BxC stem cells, and mesenchymal stem cells derived from the same tissue as the tissue from which the induced pluripotent stem cells used for the production of BxC stem cells were derived, and the results are shown in Table 1. A comparison was made of the expression levels of genes, and the results are shown in Table 2.

TABLE 1

| | | |
|---|---|---|
| Protein name | MSC | BxC |

| | | | | |
|---|---|---|---|---|
| Endostatin | 3516.6 | pg/ml | 6682.0 | pg/ml |
| Endothelin-1 | 17.0 | pg/ml | 79.4 | pg/ml |
| VEGF-A | 32.2 | pg/ml | 78.5 | pg/ml |
| Thrombospondin-2 | 21802.9 | pg/ml | 36912.7 | pg/ml |
| PIGF | 5.8 | pg/ml | 9.4 | pg/ml |
| PDGF-AA | 29.2 | pg/ml | 328.4 | pg/ml |
| beta-NGF | 10.5 | pg/ml | 17.3 | pg/ml |
| HB-EGF | 3.9 | pg/ml | 15.7 | pg/ml |

TABLE 2

| | | | Expression |
|---|---|---|---|
| | | | level |
| | | | relative |
| | Gene | | to MSCs |
| NO | name | Explanation | (fold change) |
| 1 | ANKRD1 | ankyrin repeat domain 1 | 55.1 |
| 2 | CPE | carboxypeptidase E | 38.8 |
| 3 | NKAIN4 | sodium/potassium transporting | 34.3 |
| | | ATPase interacting 4 | |
| 4 | LCP1 | lymphocyte cytosolic protein 1 | 28.8 |
| 5 | CCDC3 | coiled-coil domain containing 3 | 25.4 |
| 6 | MAMDC2 | MAM domain containing 2 | 19.9 |
| 7 | CLSTN2 | calsyntenin 2 | 19.1 |
| 8 | SFTA1P | surfactant associated 1, pseudogene | 16.4 |
| 9 | EPB41L3 | erythrocyte membrane protein band | 16.1 |
| | | 4.1 like 3 | |
| 10 | PDE1C | phosphodiesterase 1C | 15.4 |
| 11 | EMILIN2 | elastin microfibril interfacer 2 | 15.1 |
| 12 | SULT1C4 | sulfotransferase family 1C member 4 | 13.8 |
| 13 | TRIM58 | tripartite motif containing 58 | 13.2 |
| 14 | DENND2A | DENN domain containing 2A | 12.5 |
| 15 | CADM4 | cell adhesion molecule 4 | 12.2 |
| 16 | AIF1L | allograft inflammatory factor 1 like | 11.6 |
| 17 | NTM | neurotrimin | 11.5 |
| 18 | SHISA2 | shisa family member 2 | 11.0 |
| 19 | RASSF4 | Ras association domain family | 10.9 |
| | | member 4 | |
| 20 | ACKR3 | atypical chemokine receptor 3 | 10.1 |

Thereafter, the BxC stem cells were further cultured in a culture medium containing High glucose DMEM (Gibco, USA), 10% FBS (HyClone, USA), and 1% MEM Non-Essential Amino Acids Solution (100×, Gibco, USA) to completely differentiate into induced pluripotent stem cell-derived mesenchymal stem cells.

### Example 2: Production of Hyaluronic Acid-Pretreated BxC Stem Cells and Isolation of Exosomes (BxC-Exo)

The induced pluripotent stem cell-derived mesenchymal stem cells (BxC) prepared in Example 1 were cultured for 24 hours in high-glucose DMEM medium supplemented with 10% fetal bovine serum, 1% MEM non-essential amino acids solution, and 40 μg/ml hyaluronic acid, thereby producing hyaluronic acid-pretreated BxC stem cells.

After completion of the culturing, the hyaluronic acid-pretreated BxC stem cells were washed and cultured for additional 72 hours in a culture medium supplemented with 10% exosome-free FBS.

After 72 hours of culturing, the culture pretreated with the pretreatment substance was harvested and centrifuged at 300×g for 10 minutes to remove remaining cells and cell debris. Subsequently, the supernatant was taken, filtered through a 0.22-μm filter, and centrifuged at 10,000×g and 4° C. for 70 minutes by using a high-speed centrifuge. Then, the supernatant thus formed was taken and centrifuged at 100,000×g and 4° C. for 90 minutes by using an ultracentrifuge. After the removal of the supernatant, the exosomes remaining in the bottom layer were diluted in PBS to isolate exosomes derived from hyaluronic acid-pretreated BxC stem cells (hereinafter, BxC-Exo), which were used in the subsequent experiments.

### Example 3: Wound Healing Experiments in Burn Injury-Induced Animal Models

### 3-1. Examination of Wound Healing Rates Through Naked-Eye Observation of Wound Areas

To examine whether the composition of the present disclosure promoted wound healing in animal models, burn injury-induced animal models were treated with BxC-Exo produced in Example 2, and then the degree of wound healing over time was compared and observed.

Specifically, BALB/c mice were anesthetized using isoflurane, and the dorsal hairs of the anesthetized mice were shaved using a shaving cream. Thereafter, a burn of the same size was induced for each mouse by using an iron with a diameter of 1 cm. The mice were grouped into test and control groups, which were then subjected to subcutaneous (SC) injection of test and control substances, respectively. The test group was administered 200 ug of BxC-Exo per mouse, and the control group was administered PBS.

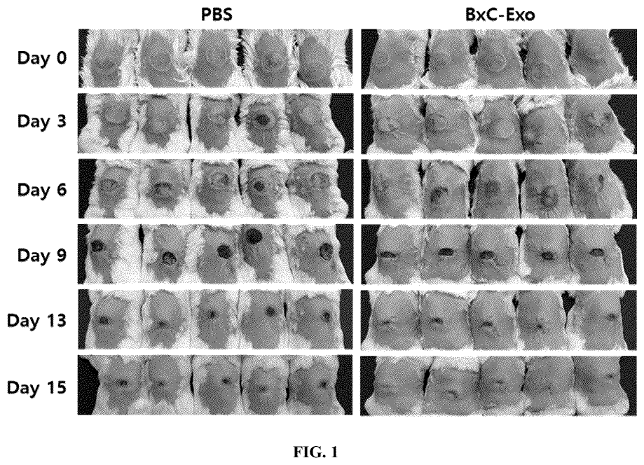

The degree of wound healing was observed for 15 days for each subject that had been administered individual substance, and Tegaderm (3M) was attached to the wound of each subject to prevent contamination. Particularly, the wound area was calculated for each subject, with 0% for the area immediately after wound formation and 100% at the time of complete wound healing, to measure the mean and standard error of the mean (SEM) of the wound healing rate over time. The results are shown in FIGS. 1 and 2 and Table 3.

TABLE 3

| Wound enclosure | | Day | Day | Day | Day | Day | Day |
|---|---|---|---|---|---|---|---|
| rate (%) | | 0 | 3 | 6 | 9 | 13 | 15 |
| Control group | Mean | 0 | 5.6 | 5.2 | 19.3 | 63.6 | 89.3 |
| (PBS) | SEM | 0 | 3.9 | 2.0 | 6.4 | 1.7 | 4.2 |
| Test group | Mean | 0 | 11.3 | 29.7 | 80.9 | 96.0 | 99.4 |
| (BxC-Ex0) | SEM | 0 | 6.6 | 6.3 | 1.9 | 0.5 | 0.2 |

As a result of measurement, the control group administered PBS showed a wound healing rate of at most 20% until Day 9, but the test group administered BxC-Exo showed a wound healing rate of 80% on Day 9 and 96% on Day 13. It was therefore confirmed that BxC-Exo can promote rapid wound healing.

### 3-2. Histopathological Staining Analysis of Wound Area

After the naked-eye observation according to Example 3-1, wound tissues collected from the corresponding animal models were subjected to special staining and histopathological analysis.

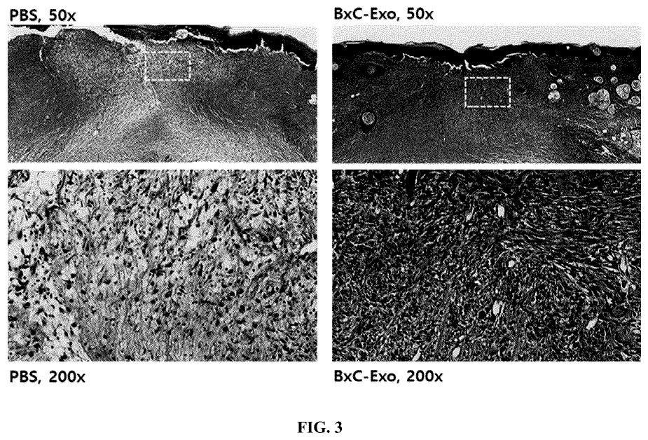

Specifically, the naked-eye observation of the wounds of the animal model BALB/c mice undergoing burn induction and administered the control substance (PBS) or test substance (BxC-Exo) were terminated on Day 15, and then the wound tissue from each subject was collected. The collected tissues were fixed with 10% formaldehyde and embedded in paraffin, and 4-um thick tissue sections were obtained. Thereafter, the tissues were observed by staining elastin fibers in black and collagen fibers in red through Van Gieson staining on the tissue sections. The results are shown in FIG. 3.

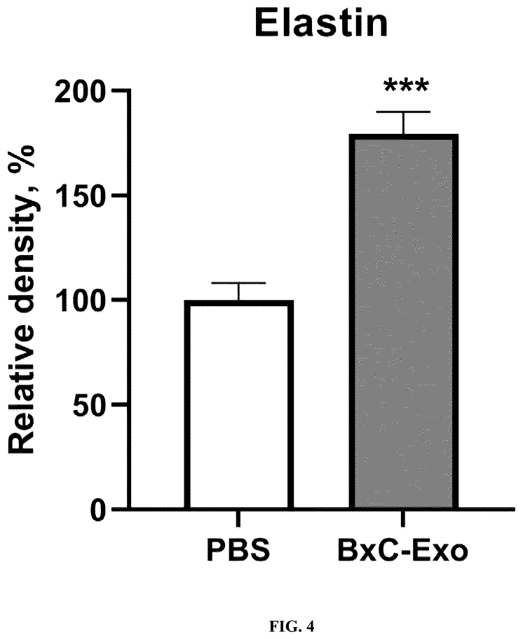

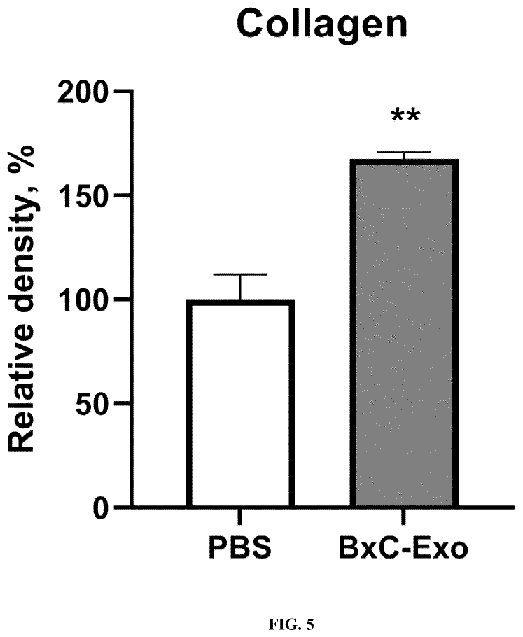

On the 400×g magnification image of each stained tissue sample, three regions of interest (ROIs) of the same size were set per slide of the burn wound lesion, and then the densities of the elastin fibers (black) and collagen fibers (red) were separately measured using the Image J program. Particularly, the burn wound lesion was confined to the dermal layer where skin appendages such as hair follicles are absent. After the density of each type of fibers measured in the control group administered PBS was set to 100%, the mean and standard error of the mean (SEM) of the relative density of each type of fibers in the test group administered BxC-Exo were calculated. The results are shown in FIGS. 4 and 5 Tables 4 and 5.

TABLE 4

| Elastin relative | Control group | Test group |
|---|---|---|
| density (%) | (PBS) | (BxC-Exo) |
| Mean | 100 | 179.6 |
| SEM | 8.0 | 10.3 |

TABLE 5

| Collagen relative | Control group | Test group |
|---|---|---|
| density (%) | (PBS) | (BxC-Exo) |
| Mean | 100 | 167.6 |
| SEM | 12.1 | 3.1 |

As a result of relative density calculation, the density of elastin fibers was 179.6% and the density of collagen fibers was 167.6% in the test group administered BxC-Exo, showing histologically superior wound healing compared with the control group. Taken these results together with the observation results of wound healing rates in Example 3-1, the degree of wound healing in the test group was much superior in terms of histology, despite an apparent difference in wound closure rate of approximately 10% between the test and control groups.

### Example 4: Would Healing of Human Dermal Fibroblasts

To examine whether the composition of the present disclosure promoted the wound healing of human dermal fibroblasts (HDF), a comparison was made of the wound healing rate 24 hours after the treatment of scratch wound-induced human dermal fibroblasts with the exosomes (BxC-Exo) produced in Example 2 (scratch assay).

Specifically, human dermal fibroblasts were seeded at 35,000 cells per well into a 12-well plate and cultured in a basic medium at 37° C. and 5% CO₂ for 16 hours or longer for cell adhesion. Particularly, the composition of the basic medium was high-glucose DMEM, 10% FBS, and 100 U/mL penicillin-streptomycin.

When the cell confluency reached 90% or more for each well, a scratch was made using a 100-p micropipette tip, and the cells were cultured for 24 hours in serum-free DMEM medium after the treatment with the test substance (1%, 5%, or 10% v/v BxC-Exo) or the control substance (PBS or 100 ug/mL hyaluronic acid (HA)). The groups treated with the test substance at different concentrations were set as test groups, the PBS treatment group was set as the negative control, and the HA treatment group was set as the positive control.

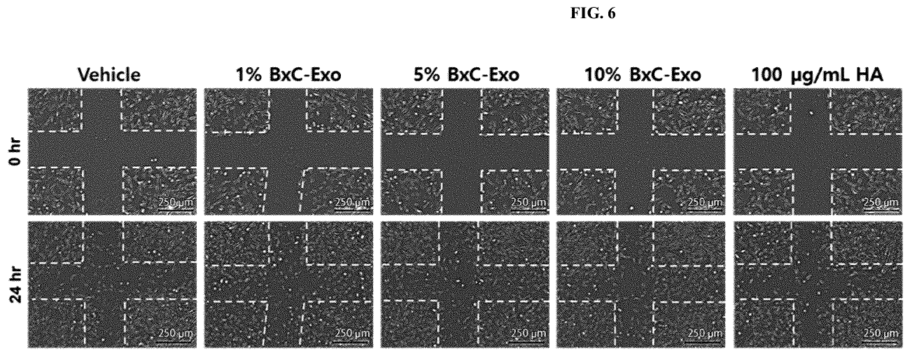

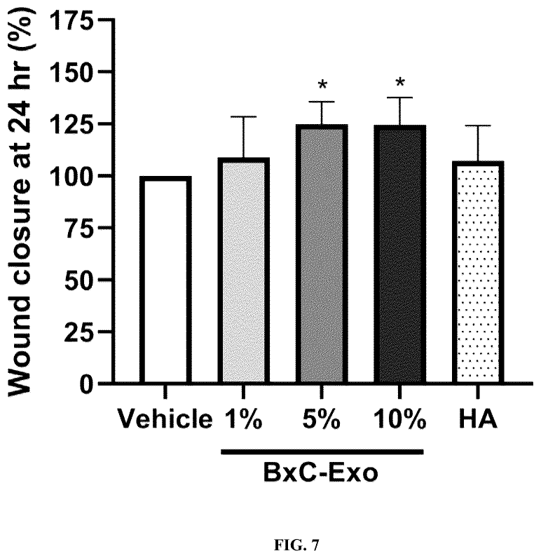

The degree of wound healing in each group was observed after 24-hour culturing, and the results are shown in FIG. 6. The wound healing rate for each well was measured using the Image J program and then calculated as the mean and standard error of the mean (SEM) of relative values in the test groups and the positive control group, with the mean value of the negative control group set as 100%. The results are shown in FIG. 7 and Table 6.

TABLE 6

| Relative | Negative | Test groups | | | Positive |
|---|---|---|---|---|---|
| wound | control | 1% v/v | 5% v/v | 10% v/v | control |
| healing | group | BxC- | BxC- | BxC- | group |
| rate (%) | (PBS) | Exo | Exo | Exo | (HA) |
| Mean | 100.0 | 108.9 | 124.9 | 124.7 | 107.3 |
| SEM | 0.0 | 7.1 | 4.0 | 4.7 | 6.2 |

As a result of calculating the relative values of wound closure degree, the wound healing rate was increased by about 7.3% in the positive control group compared with the negative control group, and the test groups showed an increasing proliferation rate of human dermal fibroblasts with the increasing concentration of drug treatment. However, there was no significant difference between the 5% v/v treatment group and the 10% v/v treatment group, and the two treatment groups showed an increase of about 25% in wound healing rate compared with the negative control.

### Example 5: Gene Expression Analysis of Human Dermal Fibroblasts

To examine whether the composition of the present disclosure promoted the expression of genes encoding wound healing factors (EGF and IGF1) and genes encoding extracellular matrix (ELN, FN1, and COL1A1) produced from fibroblasts, a comparison was made of the expression level of each gene 24 hours after the treatment of human dermal fibroblasts with exosomes (BxC-Exo) produced in Example 2.

Specifically, human dermal fibroblasts were seeded at 20,000 cells per well into a 12-well plate and cultured in a basic medium at 37° C. and 5% CO₂ for 16 hours or longer for cell adhesion. Particularly, the composition of the basic medium was the same as that of the basic medium in Example 4.

Thereafter, the cells were cultured for 24 hours in serum-free DMEM medium after the treatment with the test substance (100 ug/mL BxC-Exo) or the control substance (PBS or 100 ug/mL hyaluronic acid (HA)). Particularly, the BxC-Exo treatment group was set as an test group, the PBS treatment group was set as the negative control, and the HA treatment group was set as the positive control.

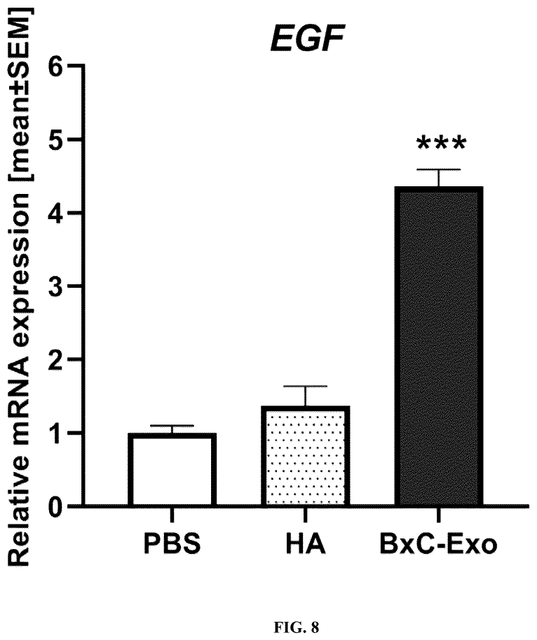

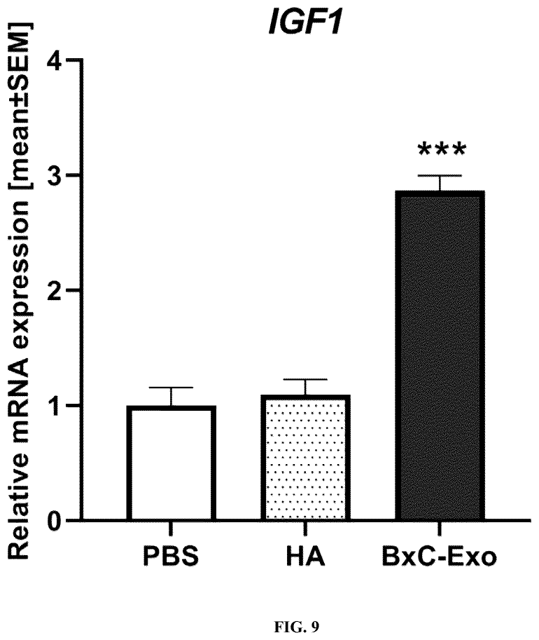

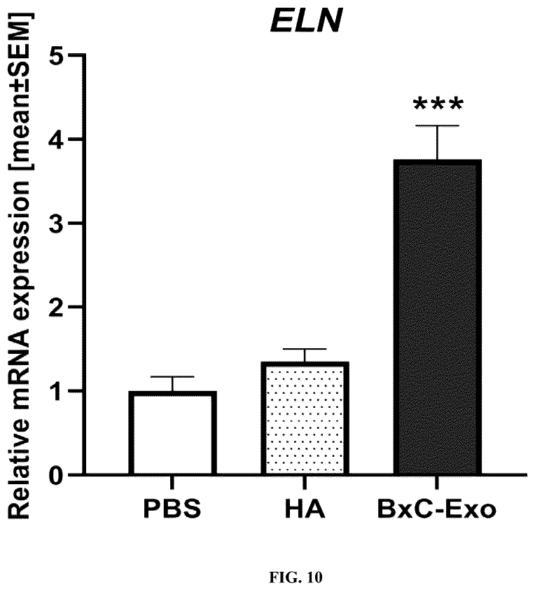

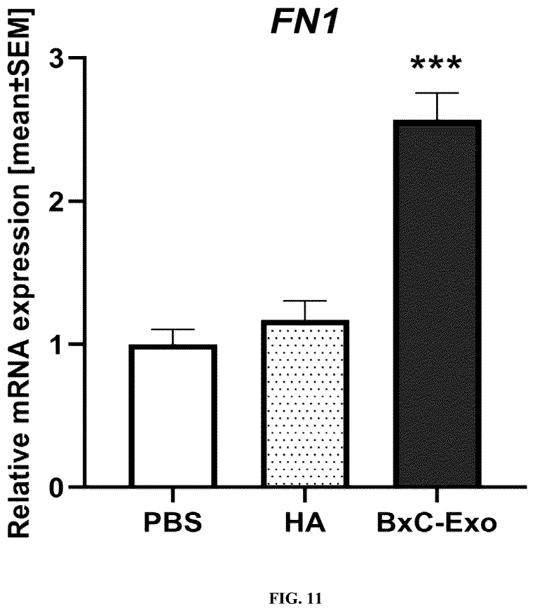

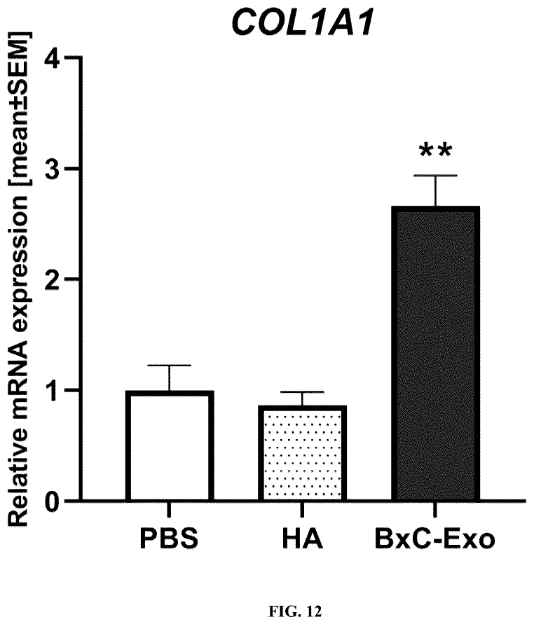

After the completion of 24-hour culturing, the cells were collected from each medium, and total RNA was extracted from each medium by using TRIzol™ (Invitrogen). Thereafter, the mRNA expression levels of the EGF, IGF1, ELN, FN1, and COL1A1 genes were measured for an equal amount of total RNA extracted from each medium through real-time PCR (Applied Biosystems) analysis. The measurement values for each gene were normalized based on the GAPDH expression level according to the 2^{-ΔΔCt} technique and then quantified relative to the quantity in the negative control group, which was set as 1. The means and standard error of the mean (SEM) of the quantity with respect to the expression levels of respective genes are shown in FIG. 8 and Table 7, FIG. 9 and Table 8, FIG. 10 and Table 9, FIG. 11 and Table 10, and FIG. 12 and Table 11.

TABLE 7

| Relative expression | Negative control | Positive control | Test group |
|---|---|---|---|
| level (EGF) | group (PBS) | group (HA) | (BxC-Exo) |
| Mean | 1.0 | 1.4 | 4.4 |
| SEM | 0.1 | 0.3 | 0.2 |

TABLE 8

| Relative expression | Negative control | Positive control | Test group |
|---|---|---|---|
| level (IGF1) | group (PBS) | group (HA) | (BxC-Exo) |
| Mean | 1.0 | 1.1 | 2.9 |
| SEM | 0.2 | 0.1 | 0.1 |

TABLE 9

| Relative expression | Negative control | Positive control | Test group |
|---|---|---|---|
| level (ELN) | group (PBS) | group (HA) | (BxC-Exo) |
| Mean | 1.0 | 1.3 | 3.8 |
| SEM | 0.2 | 0.1 | 0.4 |

TABLE 10

| Relative expression | Negative control | Positive control | Test group |
|---|---|---|---|
| level (FN1) | group (PBS) | group (HA) | (BxC-Exo) |
| Mean | 1.0 | 1.2 | 2.6 |
| SEM | 0.1 | 0.1 | 0.2 |

TABLE 11

| Relative expression | Negative control | Positive control | Test group |
|---|---|---|---|
| level (COL1A1) | group (PBS) | group (HA) | (BxC-Exo) |
| Mean | 1.0 | 0.9 | 2.7 |
| SEM | 0.2 | 0.1 | 0.3 |

As a result of quantification, the test group, compared with the negative group, showed a 4.4-fold increase and a 2.9-fold increase in expression of the wound healing factors EGF and IGF1, respectively, and a 3.8-fold increase, a 2.6-fold increase, and a 2.7-fold increase in expression of ELN, FN1, and COL1A1, encoding the extracellular matrices elastin, fibronectin, and collagen, respectively. It was therefore found that BxC-Exo can produce both a healing effect on wound lesions and a restoration effect on extracellular environments.

What is claimed is:

1. A method for preparing a pharmaceutical composition for treatment of wounds, the method comprising:
a first culturing step of culturing induced pluripotent stem cells in a medium;
a selective culturing step of separating SSEA-4(−) cells not expressing SSEA-4 protein from the cultured induced pluripotent stem cells and culturing the SSEA-4(−) cells to allow the SSEA-4(−) cells to differentiate into BxC stem cells;
a second culturing step of culturing BxC stem cells to allow the BxC stem cells to differentiate into mesenchymal stem cells;
a pretreatment step of pretreating the mesenchymal stem cells with hyaluronic acid;
a producing step of culturing the pre-treated mesenchymal stem cells to produce exosomes; and
an isolating step of isolating exosomes from the mesenchymal stem cells or a culture thereof.

2. A method for treating a wound, the method comprising:
administering to a subject in need thereof a composition comprising exosomes isolated from induced pluripotent stem cell (iPSC)-derived mesenchymal stem cells (MSCs) pretreated with a pretreatment substance.

3. The method of claim 2, wherein the induced pluripotent stem cell-derived mesenchymal stem cells are differentiated from a progenitor cell of induced pluripotent stem cell-derived mesenchymal stem cells not expressing stage-specific embryonic antigen 4 (SSEA-4) protein.

4. The method of claim 2, wherein the induced pluripotent stem cell is a human-derived induced pluripotent stem cell.

5. The method of claim 2, wherein the pretreatment substance is hyaluronic acid.
